(19) United States
(12) Patent Application Publication
(10) Pub. No.: US 2026/0306352 A1
(43) Pub. Date: Oct. 8, 2026

(54) SYSTEM AND METHOD FOR ENHANCING VENOUS CIRCULATION IN A LIMB OF A USER

(71) Applicants: Evans Tachie-Menson, Katy, TX (US); Kingsley Essel Arthur, Teshie- Nungua, Accra (GH)

(72) Inventors: Evans Tachie-Menson, Katy, TX (US); Kingsley Essel Arthur, Teshie-Nungua (GH)

(21) Appl. No.: 19/326,994

(22) Filed: Sep. 12, 2025

Related U.S. Application Data
(60) Provisional application No. 63/782,468, filed on Apr. 2, 2025.
(60) Provisional application No. 63/817,321, filed on Jun. 4, 2025.

(51) Int. Cl. A61H 23/02; A61N 1/36

(52) U.S. Cl. CPC A61H 23/02; A61H 2201/0107; A61H 2201/164; A61H 2201/165; A61H 2201/5071; A61H 2201/5089; A61H 2201/5097; A61H 2205/12; A61H 2209/00; A61H 2230/255; A61N 1/36031

(57) ABSTRACT

An apparatus enhances venous circulation in a limb using a first sensor set that detects load, a second sensor set that optically detects a blood circulation level, and a third sensor set that detects movement and orientation. A controller determines a venous-pooling index from the sensed load, blood circulation level, and motion data and activates a haptic actuator to deliver external stimulation when the index exceeds a threshold. In some embodiments the controller applies filtering and optional machine-learning personalization to adjust stimulation based on user activity or environmental conditions, including zero-gravity operation.

### CROSS REFERENCE

This non-provisional application claims the benefit under 35 U.S.C. § 119 (e) of U.S. Provisional Patent Applications Ser. No. 63/817,321, entitled “Wearable Dynamic Foot Pump Assessment and Stimulation System (DFPAS)” filed on Jun. 4, 2025 and Ser. No. 63/782,468 entitled “Dynamic Foot Pump Assessment System (DFPAS) for Enhancing Circulatory Health in Microgravity and Clinical Environments” dated 2 Apr. 2025.

### TECHNICAL FIELD

The present disclosure relates to the field of human diagnostics. More particularly, the present disclosure relates to an apparatus for detecting and enhancing venous circulation in a limb of a user.

### BACKGROUND

Background description includes information that may be useful in understanding the present invention. It is not an admission that any of the information provided herein is prior art or relevant to the presently claimed invention, or that any publication specifically or implicitly referenced is prior art.

A problem of venous pooling in lower limbs of a user, particularly in feet and calves, pose significant health risks such as edema, orthostatic intolerance, and deep-vein thrombosis (DVT). These health-related risks arise due to inadequate venous circulation and occur especially when the user has to either stand for a prolonged period, is immobile, or is exposed to a zero-gravity environment.

Traditional strategies for countering the problem of the venous pooling are limited in their effectiveness and are inadequate to provide real time therapeutic intervention as these strategies involve only passive monitoring of the user and merely collect physiological user data without providing an immediate corrective action, or are related to using open loop compression devices that operate on fixed duty cycles and do not adapt to activity levels of the user. These limitations render the traditional strategies ineffective to be used in dynamic environments such as in a spaceflight and for prolonged daily use by the user.

For example, prior systems such as those described in US Patent Publication No. 2020/0178813A1 focus on venous pressure estimation for electrical stimulation but lack integrated multi-sensor fusion and adaptive indexing for zero-gravity conditions. Similarly, wearable sensors in publications like those in Nature (2024) assess arterial/venous competence but do not compute a dynamic venous-pooling index or provide haptic feedback based on real-time AI-driven decisions.

There is therefore a need to provide an improved mechanism to counter the problem of venous pooling in the lower limbs of the user.

### OBJECTS OF THE PRESENT DISCLOSURE

Some of the objects of the present disclosure, which at least one embodiment herein satisfies are as listed herein below.

An object of the present disclosure is to provide an apparatus for enhancing venous return/circulation in a limb of a user.

Another object of the present disclosure is to determine a venous-pooling index and to instruct a haptic actuator to deliver an external stimulation to the limb when the venous-pooling index exceeds a pre-defined threshold.

Another object of the present disclosure is to use the apparatus in an active user mode to augment voluntary movement of the user through the external stimulation generated by the haptic actuator.

Another object of the present disclosure is to use the apparatus in a passive user mode to activate the haptic actuator to generate the external stimulation when the user is immobile or when the user is present in a zero-gravity environment.

Another object of the present disclosure is to incorporate artificial intelligence or machine learning models for processing sensor data, predicting venous stasis trends, and personalizing stimulation patterns.

Another object of the present disclosure is to enable bilateral synchronization between multiple apparatuses for assessing symmetry in venous circulation across limbs.

### SUMMARY

An aspect of the present disclosure pertains to an apparatus for enhancing venous circulation in a limb of a user. The apparatus includes a first set of sensors that are configured to be placed in contact with the limb of the user. The first set of sensors detect an amount of load that is exerted by the limb of the user. The apparatus includes a second set of sensors configured to be placed in contact with a portion of skin of the limb of the user. The second set of sensors optically detect a blood circulation level corresponding to the limb of the user. The apparatus includes a third set of sensors configured to be placed in contact with the limb of the user. The third set of sensors detect movement and orientation of the limb of the user. The apparatus includes a haptic actuator configured to be placed in contact with the limb of the user. The haptic actuator generates an external stimulation. The apparatus includes a controller which is in communication with the first set of sensors, the second set of sensors, the third set of sensors, and the haptic actuator. The controller is configured to determine a venous-pooling index based on the amount of load that is exerted by the limb of the user, the blood circulation level corresponding to the limb of the user, and the movement and orientation of the limb of the user. Further, the controller is configured to instruct the haptic actuator to deliver the external stimulation to the limb when the venous-pooling index exceeds a pre-defined threshold, to enhance the venous circulation in the limb of the user.

In an embodiment, the controller incorporates artificial intelligence (AI) or machine learning (ML) models to process sensor data, predict trends in venous stasis, and adapt stimulation in real-time.

In an embodiment, the apparatus supports bilateral synchronization with a paired device on another limb for comparative analysis.

Another aspect of the present disclosure pertains to a footwear comprising an outer sole, an inner sole, and the apparatus placed between the outer sole and the inner sole of the footwear such that the limb of the user is placed on the apparatus placed beneath the inner sole. The apparatus includes the first set of sensors that are configured to be placed in contact with the limb of the user. The first set of sensors detect the amount of load that is exerted by the limb of the user. The apparatus includes the second set of sensors configured to be placed in contact with the portion of skin of the limb of the user. The second set of sensors optically detect a blood circulation level corresponding to the limb of the user. The apparatus includes the third set of sensors configured to be placed in contact with the limb of the user. The third set of sensors detect movement and orientation of the limb of the user. The apparatus includes the haptic actuator which is configured to be placed in contact with the limb of the user. The haptic actuator generates an external stimulation. The apparatus includes the controller which is in communication with the first set of sensors, the second set of sensors, the third set of sensors, and the haptic actuator. The controller is configured to determine a venous-pooling index based on the amount of load that is exerted by the limb of the user, the blood circulation level corresponding to the limb of the user, and the movement and orientation of the limb of the user. Further, the controller is configured to instruct the haptic actuator to deliver the external stimulation to the limb when the venous-pooling index exceeds the pre-defined threshold, to enhance the venous circulation in the limb of the user.

Another aspect of the present disclosure pertains to a method for enhancing the venous circulation in the limb of the user. The method includes detecting, by the first set of sensors, the amount of load exerted by the limb of the user. The first set of sensors are configured to be placed in contact with the limb of the user. The method includes detecting, by the second set of sensors, a blood circulation level corresponding to the limb of the user. The second set of sensors are configured to be placed in contact with the portion of skin of the limb of the user to optically detect the blood circulation level. The method includes detecting, by the third set of sensors, movement and orientation of the limb of the user. The third set of sensors are configured to be placed in contact with the limb of the user. The method includes generating, by the haptic actuator, an external stimulation. The haptic actuator is configured to be placed in contact with the limb of the user. The method includes determining, by the controller, the venous-pooling index based on the amount of load that is exerted by the limb of the user, the blood circulation level corresponding to the limb of the user, and the movement and orientation of the limb of the user. The controller is configured to instruct the haptic actuator to generate the external stimulation for the limb when the venous-pooling index exceeds the pre-defined threshold, to enhance the venous circulation in the limb of the user.

In an embodiment, the first set of sensors comprises one or more pressure sensors.

In an embodiment, a preload assembly is configured to apply a contact force to the one or more pressure sensors which are placed in contact with the limb of the user, to capture pressure measurements corresponding to the limb in a zero-gravity condition.

In an embodiment, under the zero-gravity condition, the contact force applied to the one or more pressure sensors ranges from 0.5 newtons to 2 newtons.

In an embodiment, the controller is configured to operate in an active user mode, and a passive user mode associated with the user, wherein during the operation of active user mode, the controller is configured to augment voluntary movement of the user through the external stimulation generated by the haptic actuator. During the operation of passive user mode, the controller is configured to activate the haptic actuator to generate the external stimulation when the user is immobile or when the user is present in the zero-gravity environment.

In an embodiment, the second set of sensors comprises one or more optical sensors.

In an embodiment, the third set of sensors comprises one or more motion sensors.

In an embodiment, the limb is a lower limb of the user and corresponds to a foot of the user.

In an embodiment, the haptic actuator is positioned under a first metatarsal head and a second metatarsal head of the foot of the user.

In an embodiment, the apparatus includes any or a combination of a wearable cuff, a footwear and a sock.

In an embodiment, the apparatus is implemented in modular form factors including ankle cuffs, adhesive patches, leg bands, calf straps, or chair-mounted controllers, and may support wireless communication via Bluetooth Low Energy (BLE) for data transmission to mobile devices or cloud systems.

### BRIEF DESCRIPTION OF DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure. The diagrams are for illustration only, which thus is not a limitation of the present disclosure.

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates an exemplary plan view of an apparatus for enhancing a venous circulation in a limb of a user, in accordance with an embodiment of the present disclosure.

FIG. 2 illustrates an exemplary exploded view of an insole of a footwear, in accordance with an embodiment of the present disclosure;

FIG. 3 illustrates an exemplary footwear having sensing and stimulation elements, in accordance with an embodiment of the present disclosure.

FIG. 4 illustrates the apparatus embedded in a sock, in accordance with an embodiment of the present disclosure;

FIG. 5 illustrates the apparatus embedded in different configurations, in accordance with an embodiment of the present disclosure;

FIG. 6 illustrates the apparatus being used for enhancing the venous circulation in the limb of the user when the user is sitting, in accordance with an embodiment of the present disclosure; and FIG. 7 illustrates a method for enhancing the venous circulation in the limb of the user, in accordance with an embodiment of the present disclosure.

FIG. 8 illustrates a block diagram of a firmware architecture for a controller of the apparatus, including data acquisition, filtering, AI decision logic, and wireless communication layers, in accordance with an embodiment of the present disclosure.

FIG. 9 illustrates a flow chart for calculating a venous-pooling index using a weighted algorithm and machine learning model, in accordance with an embodiment of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

The ensuing description provides exemplary embodiments only and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the disclosure as set forth.

Specific details are given in the following description to provide a thorough understanding of the embodiments. However, it will be understood by one of ordinary skill in the art that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail to avoid obscuring the embodiments.

Also, it is noted that individual embodiments may be described as a process that is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination can correspond to a return of the function to the calling function or the main function.

The word “exemplary” and/or “demonstrative” is used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive in a manner similar to the term “comprising” as an open transition word without precluding any additional or other elements.

Reference throughout this specification to “one embodiment” or “an embodiment” or “an instance” or “one instance” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terminology used herein is for the purpose of describing particular embodiments only and is not intended to be limiting of the disclosure. As used herein, the singular forms “a”, “an”, and “the” are intended to include the plural forms as well, unless the context indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

The present disclosure relates to an apparatus and a method that is used to monitor and actively enhance venous circulation in a limb of a user. More specifically, it relates to a closed loop arrangement that integrates multiple pressure sensors, optical sensors, inertial measurement sensors, and a haptic stimulator to enhance the venous circulation during a normal terrestrial gait situation and in microgravity conditions such as in a zero-gravity environment.

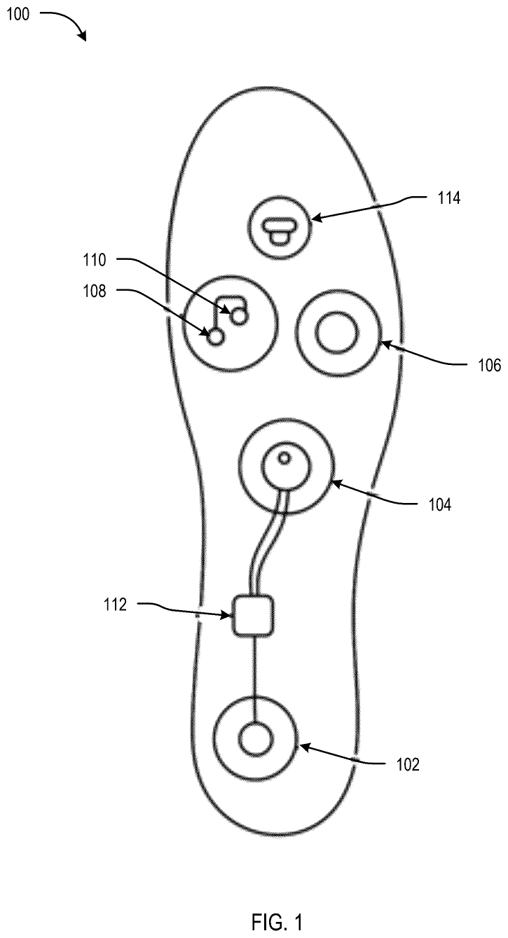

FIG. 1 illustrates an exemplary plan view of an apparatus 100 for enhancing the venous circulation in the limb of the user, in accordance with an embodiment of the present disclosure. With reference to FIG. 1, the apparatus 100 may include a first set of sensors 102, 104, and 106, a second set of sensors 108 and 110, a third set of sensors 112, and a haptic actuator 114 for generating an external stimulation to enhance the venous circulation in the limb of the user. The limb may be, for example, a lower limb of the user. The lower limb of the user may refer to, for example, a hip, a thigh, a knee, a leg, an ankle, a foot of the user.

The term user as used herein may generally refer to a human, but may also refer to an animal. In various embodiments, the user may refer to a user of the apparatus and/or a patient.

The first set of sensors 102, 104, and 106 may include one or more pressure sensors, for example, a force sensitive resistor, a piezoelectric element, a capacitive sensor, and the like. The first set of sensors 102, 104, and 106 may be placed in contact with the limb of the user. The first set of sensors 102, 104, and 106 may detect an amount of load that is exerted by the limb of the user. In an exemplary embodiment, the first set of sensors 102, 104, and 106 may be positioned under, for example, heel, medial arch, and forefoot of the lower limb (e.g., the foot) of the user.

The second set of sensors 108 and 110 may include one or more optical sensors, for example, a photoplethysmography (PPG) sensor, a Near-Infrared Spectroscopy (NIRS) sensor, and the like. The second set of sensors 108 and 110 may be placed in contact with a portion of skin of the limb of the user. The second set of sensors 108 and 110 may optically detect a blood circulation level corresponding to the limb of the user. In an exemplary embodiment, the second set of sensors may be positioned under, for example, first and second metatarsal heads of the foot of the user. As is known, the first metatarsal head is represented by an area of the foot that is present under a big toe (hallux) and bears a large portion of body weight of the user, especially during walking and running. The second metatarsal head is represented by an area of the foot that is present just next to the first metatarsal head, under a second toe, and plays a crucial role in forefoot loading.

In an embodiment, the PPG sensor may include a transducer which may be configured to emit infrared light into a tissue through the user's skin. In addition, a photodetector may be used to detect light that is reflected from the tissue. Intensity of the reflected light may correspond to a concentration of red blood cells that are present within a capillary bed. In other words, the PPG sensor may operate by directing infrared light into the skin, where variations in back scattered light may be dependent on capillary blood volume and may be measured by the photodetector and represented as a waveform or as a line tracing. As may be appreciated, flow and/or number of the red blood cells may provide an indication of a venous state of the user.

The blood circulation level may be quantified using a Perfusion Index (PI), calculated as PI=(AC/DC)×100%, where AC is a pulsatile component of the PPG signal and DC is a non-pulsatile component. The PI provides a measure of peripheral perfusion strength and is integrated into the venous-pooling index calculation.

The third set of sensors 112 may include one or more motion sensors, for example, an inertial measurement unit (IMU), an accelerometer, a gyroscope, and the like. The third set of sensors 112 may be placed in contact with the limb of the user. The third set of sensors 112 may detect movement and orientation of the limb of the user. In an exemplary embodiment, the third set of sensors may be located at an ankle region of the foot of the user. It is to be noted that the one or more motion sensors may be used when the user is either sitting, standing, or has either a stationary status or a walking status.

In another exemplary embodiment, the one or more motion sensors may include an determining an accelerometer of gait i.e., a manner or a pattern of walking or running. The gait may include a heel-strike or a toe-strike.

The apparatus 100 may include the haptic actuator 114 which may be placed in contact with the limb of the user. The haptic actuator 114 may generate an external stimulation based on a determined venous-pooling index. In an implementation, an effective position of the haptic actuator 114 may be, for example, under the first metatarsal head and the second metatarsal head of the foot of the user. In an alternative implementation, the haptic actuator may be placed under a heel of the foot.

The apparatus 100 includes a controller (not shown) which may be configured to determine the venous-pooling index using the amount of load that is exerted by the limb of the user, the blood circulation level corresponding to the limb of the user, and the movement and orientation of the limb of the user. When the venous-pooling index exceeds a pre-defined threshold, the controller may be configured to instruct the haptic actuator 114 to deliver the external stimulation to the limb to enhance the peripheral venous circulation in the limb of the user. The external stimulation may be generated in a form of, for example, vibratory pulses or as a tactile feedback.

As may be appreciated, the venous-pooling index is a quantitative representation that is used to determine an extent of blood that is accumulated (pooled) in veins, particularly in the lower limbs (e.g., the feet, the calves) of the user. The venous-pooling index may be used to assess venous circulation efficiency, detect circulatory issues, and monitor postural or gravitational effects on blood distribution in the user. Typically, the venous-pooling index may be used to evaluate, for example, problems related to venous insufficiency or autonomic dysfunction of the lower limb of the user in the zero-gravity environment, and for augmenting voluntary movement of the user through the external stimulation.

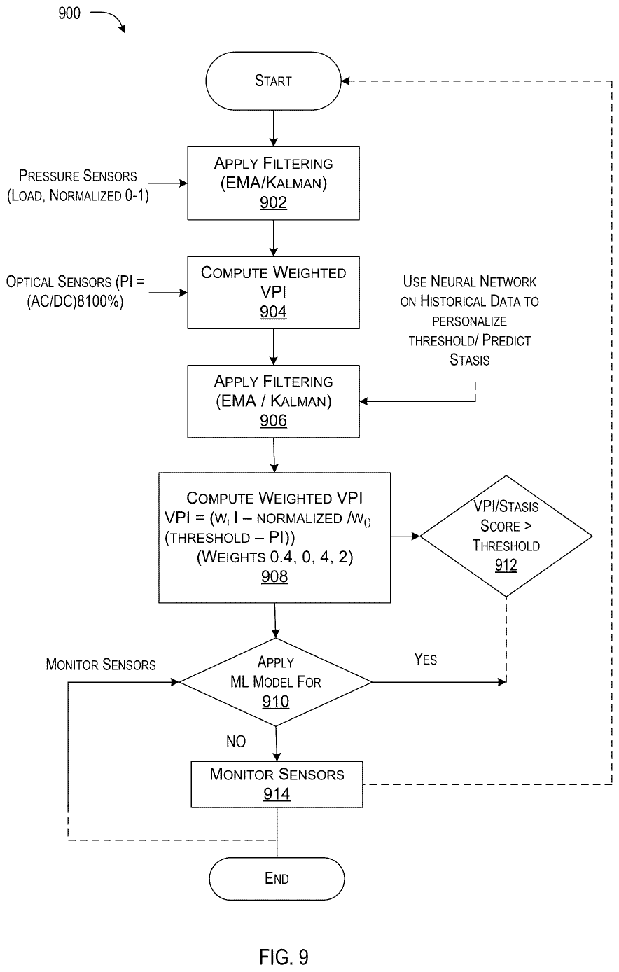

In an embodiment, the venous-pooling index (VPI) is calculated using a weighted algorithmic formula which integrates data from all the sensors. For example, VPI=w1*1−normalized_load)+w2*(blood_circulation_threshold-measured_blood_level)+w3 * (immobility_duration/max_duration), where w1, w2, w3 are weights (e.g., 0.4, 0.4, 0.2 based on empirical calibration), normalized_load is derived from pressure sensors (0-1 scale), measured_blood_level is from PPG/NIRS (e.g., perfusion index), and immobility_duration is from determined from the IMU (in seconds). The threshold may be user-personalized (e.g., 0.5) via the machine learning models trained on historical data to predict stasis trends. If VPI>threshold, stimulation is triggered. The details of this calculation are discussed in FIG. 9 in subsequent paragraphs. This formula enables adaptive, real-time decision-making, distinguishing from static prior art methods.

In some embodiments, the VPI may be referred to or computed as a Stasis Score, which is a composite metric incorporating pressure trends (AP), inactivity duration (Tinactive), and inverse perfusion (1-PI), weighted similarly to predict and mitigate venous stasis.

In an embodiment, a preload assembly may be configured to apply a contact force to the one or more pressure sensors. This may be done to capture accurate pressure measurements, corresponding to the limb of the user, in the zero-gravity condition. As may be appreciated, in the zero-gravity condition, gravity no longer causes the user to press against surfaces or exert noticeable force on them. Therefore, in the zero-gravity condition, the one or more pressure sensors may not naturally stay pressed against the skin of the user due to an absence of body weight that presses the user down. Therefore, the preload assembly may be used to apply a controlled contact force to enable capturing accurate and consistent pressure measurements. In addition, under the zero-gravity condition, the contact force applied to the one or more pressure sensors may range from, for example, 0.5 newtons to 2 newtons.

In an embodiment, the controller may be equipped with an Inter-Integrated Circuit (I²C) multiplexer or dual I²C ports for managing the first set of sensors 102, 104, and 106, the second set of sensors 108 and 110, the third set of sensors 112, and the haptic actuator 114. By way of an example, the controller may be configured inside the apparatus 100 or may be present outside the apparatus 100. The controller may be connected to the apparatus 100 through wired connections or through a wireless connection. The wireless connection may be may be executed through a wireless communication network such as, but is not limited to, a carrier network including a circuit-switched network, a public switched network, a Content Delivery Network (CDN) network, a Long-Term Evolution (LTE) network, a New Radio (NR), a Global System for Mobile Communications (GSM) network and a Universal Mobile Telecommunications System (UMTS) network, an Internet, intranets, Local Area Networks (LANs), Wide Area Networks (WANs), mobile communication networks, combinations thereof, and the like.

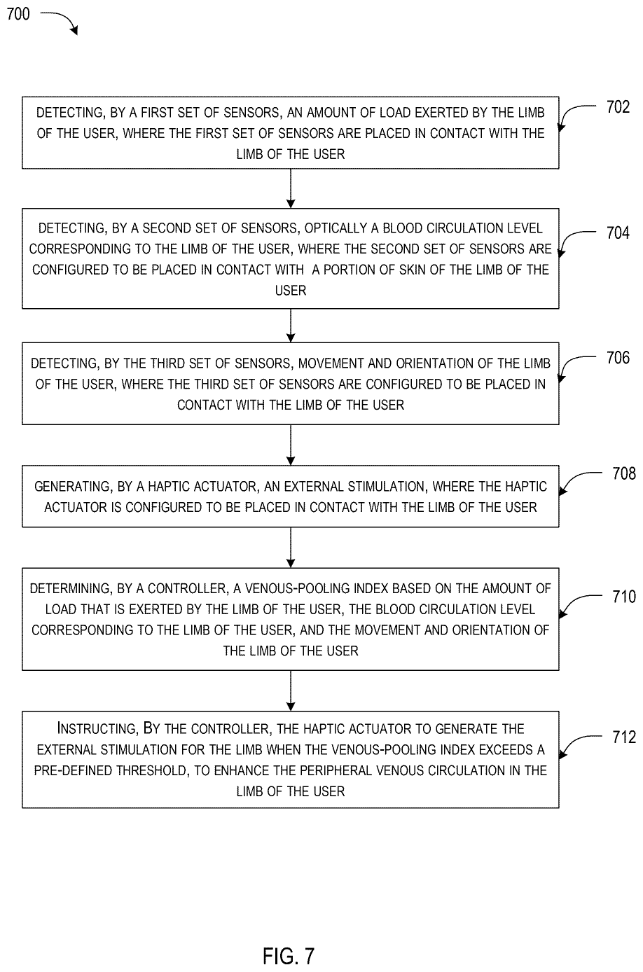

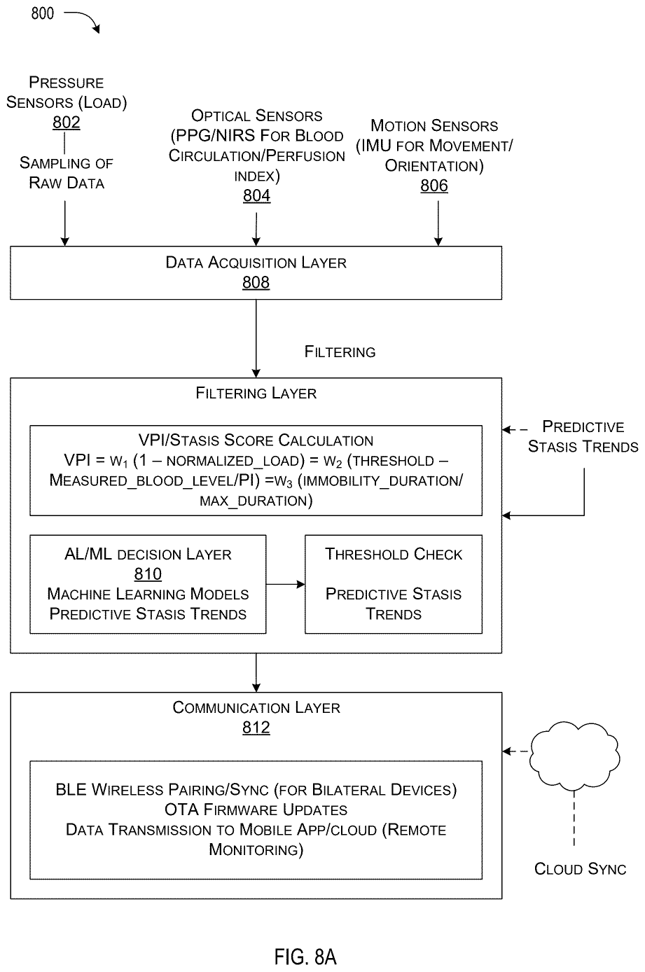

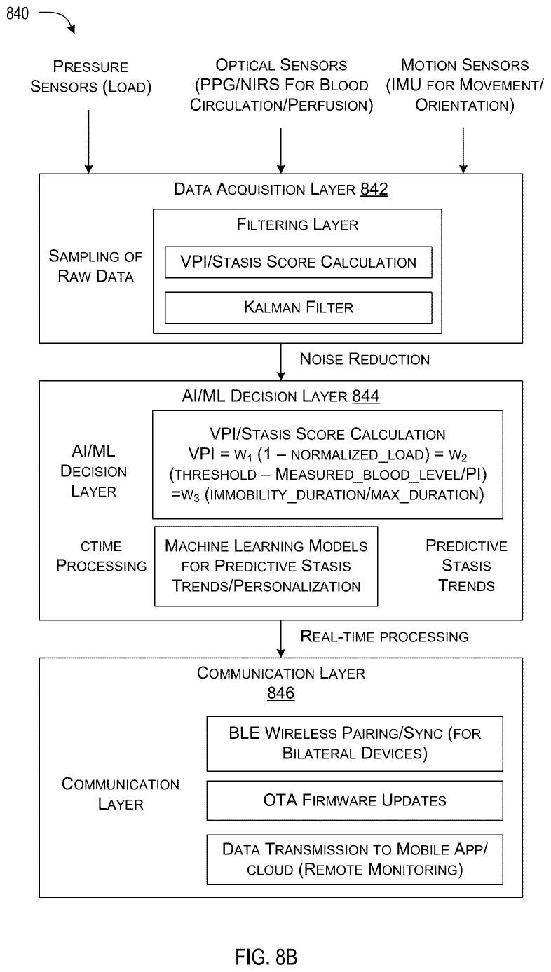

The controller may include firmware architecture comprising: a data acquisition layer for sampling sensor inputs; a filtering layer using algorithms such as Exponential Moving Average (EMA) or Kalman filters to reduce noise; an AI/ML decision layer for computing VPI and predicting trends (e.g., using lightweight models like neural networks trained on perfusion datasets); and a communication layer supporting Bluetooth Low Energy (BLE) for wireless pairing, over-the-air (OTA) updates, and data transmission to mobile apps or cloud servers for remote monitoring. Details with respect to the firmware architecture are disclosed in FIG. 8.

In an embodiment, the apparatus may support bilateral synchronization, where paired devices (e.g., left and right insoles) may communicate via the BLE to assess load symmetry or asynchronous pooling, triggering differential stimulation if asymmetry exceeds 10%.

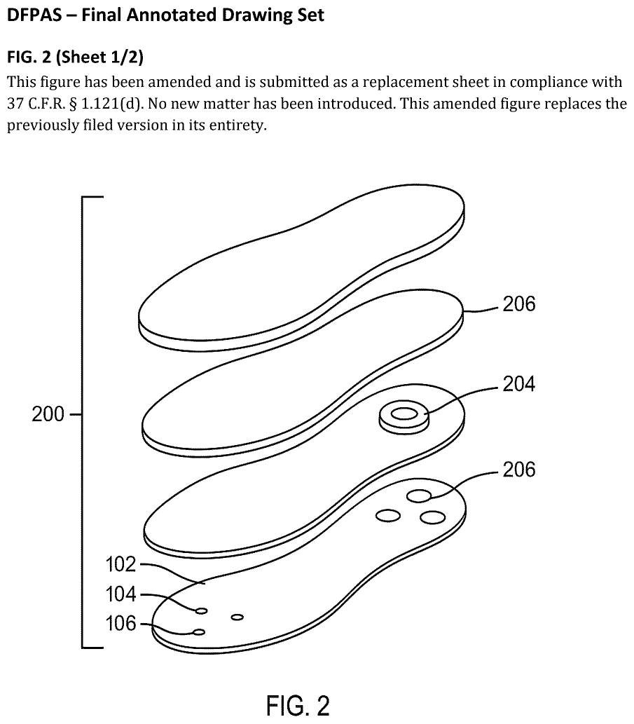

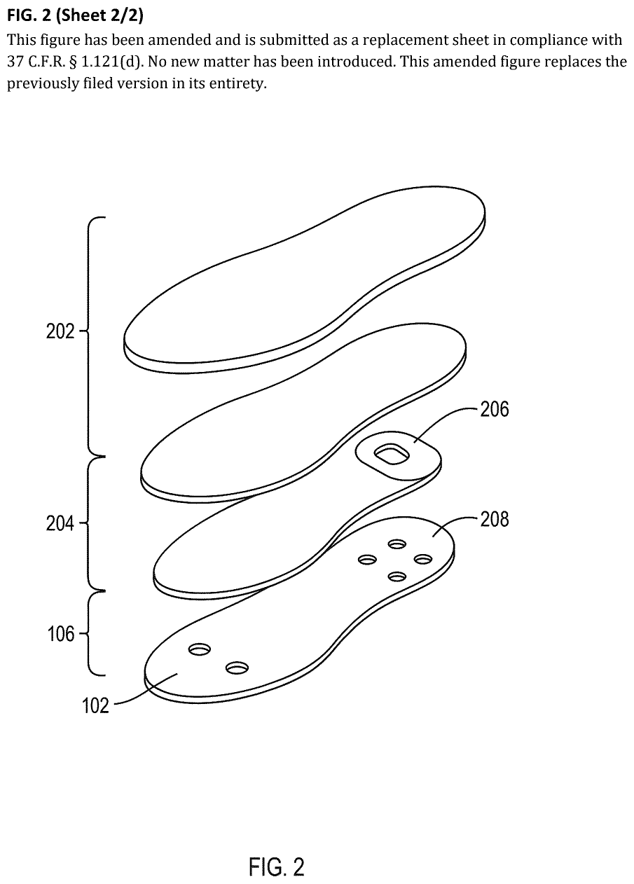

FIG. 2 illustrates an exemplary exploded view 200 of an insole of a footwear, in accordance with an embodiment of the present disclosure. As is shown in FIG. 2, an inner sole 202 of the insole directly touches the foot of the user. The inner sole 202 may be made of a soft, flexible, and breathable material and may be used for providing maximum user comfort. The inner sole 202 may facilitate to evenly distribute body pressure of the user on the footwear. The insole may have an intermediate comfort layer 204 that may be provided as a padding and may be used to hold the preload assembly. The preload assembly may be configured to apply the contact force to the one or more pressure sensors to capture pressure measurements corresponding to the limb in the zero-gravity condition.

Another layer of the insole may be a sensor routing layer 206. The sensor routing layer 206 may contain, for example, the PPG sensor, the NIRS sensor, and the like. The PPG sensor may optically detect the blood circulation level corresponding to the limb of the user.

Another layer of the insole may be a sensor embedded layer 208. The sensor embedded layer 208 may contain the one or more pressure sensors 102, 104, and 106. The one or more pressure sensors may be, for example, a force sensitive resistor (FSR), the capacitive sensor, or the piezoelectric element. The one or more pressure sensors may be likely placed at a strategic location, such as the heel or forefoot of the foot of the user, to monitor a gait phase (for example, the heel-strike or the toe-off) or a foot pressure distribution of the foot of the user. The sensor embedded layer 208 may contain multiple pressure points, sensor contacts, and ventilation holes. Circular holes as shown on the sensor embedded layer 208 may correspond to the one or more pressure sensors that may be placed under the metatarsal heads or toes of the foot of the user.

An outer sole (not shown) may form a structural base of the footwear. The outer sole may be rigid or semi rigid to maintain shape and ensure stability to the footwear. The outer sole may include holes or vents for airflow, and may enable routing of wires for connecting with other components of the system, for example, the controller.

As may be appreciated, placement of the multiple layers of the insoles, as discussed above, in the footwear may be used for determining and enhancing the venous circulation in the limb of the user, performing the gait analysis, monitoring foot pressure patterns, activating therapeutic systems, and rehabilitation and tracking sports performance of the user.

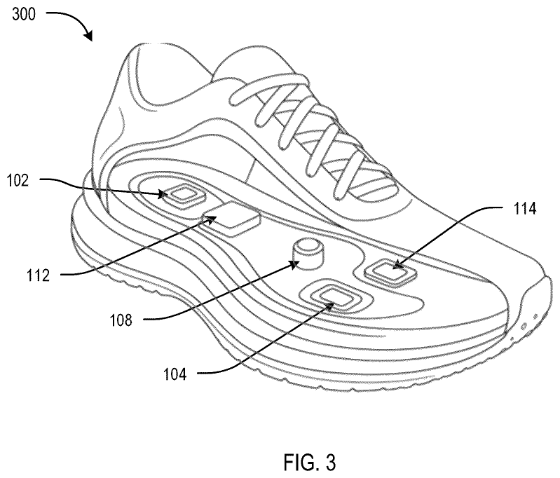

FIG. 3 illustrates an exemplary footwear 300 having sensing and stimulation elements, in accordance with an embodiment of the present disclosure. The illustrated footwear 300 may include the apparatus 100 which comprises the first set of sensors 102 and 104, the second set of sensors 108, the third set of sensors 112, and the haptic actuator 114 which are embedded and positioned within the outer sole and the inner sole of the footwear 300. The apparatus 100 embedded in the footwear 300 may enable real time monitoring of the foot pressure and may provide support in applications such as, for example, orthopedic therapy, space physiology, and the like.

The footwear 300 may include the controller that may operate in two distinct modes of the user. In the passive user mode, there is the zero-gravity environment and there is no natural weight or pressure which is present to press and push the foot down onto the first set of sensors, due to the absence of gravity. Therefore, a controlled contact force may be applied to ensure that the first set of sensors may be able to maintain a proper skin contact with the user, and provide the reliable amount of load that is exerted by the limb of the user. The controlled contact force may be maintained within a specific range between 0.5 newtons and 2 newtons such that the one or more pressure sensors may function effectively even without presence of gravitational force. Further, the controller may be configured to activate the haptic actuator to generate the external stimulation when the user is immobile or when the user is present in the zero-gravity environment.

In the active user mode, when the user is voluntarily moving (e.g., walking or exercising), the controller may work to support or enhance natural movements of the user. This may be done by activating the haptic actuator. The haptic actuator may deliver external stimulation, for example, vibration or pressure pulses that complements efforts of the user and may assist in improving the venous circulation or muscle activation in the lower limb of the user.

In some embodiments, the AI model within the controller may predict impending stasis based on trend analysis (e.g., rising immobility over 5 minutes) and adjust stimulation intensity (e.g., 1-5 Hz vibration for 30 seconds) personalized to user data.

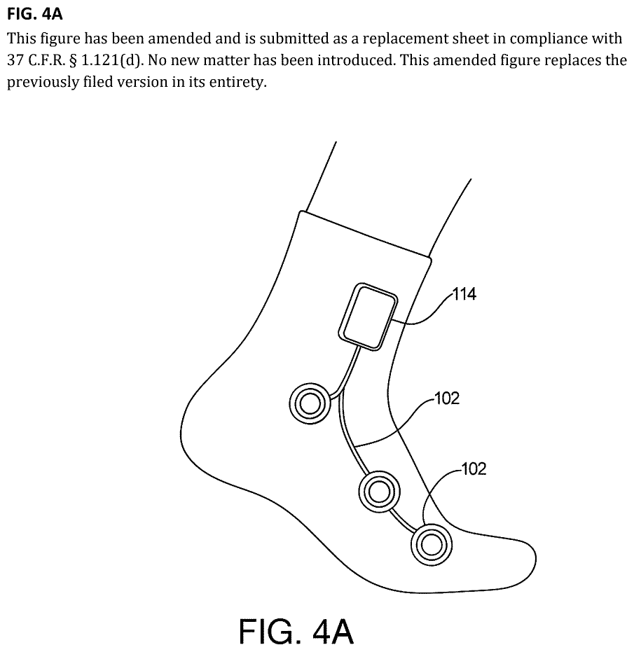

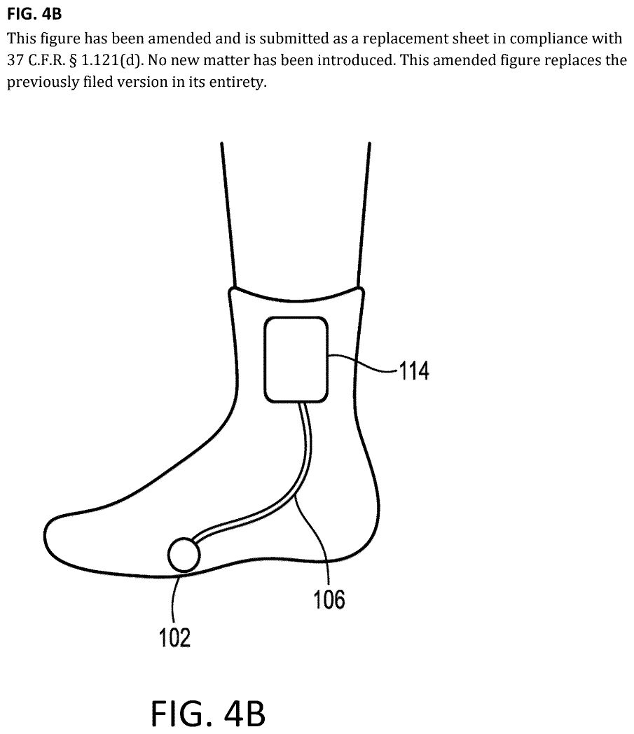

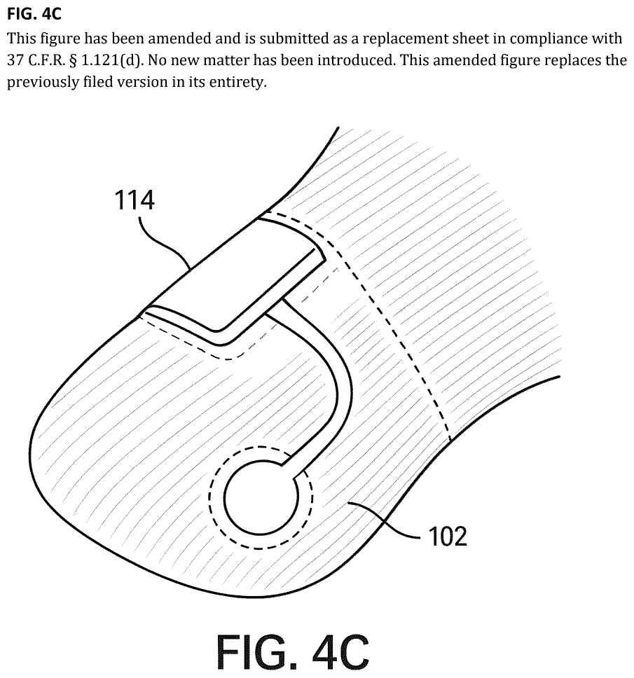

FIG. 4 illustrates the apparatus 100 embedded in a sock 400, in accordance with an embodiment of the present disclosure. In an exemplary embodiment, the apparatus 100 may be in a form of the sock which may be adapted to be worn around the foot and the ankle of the user.

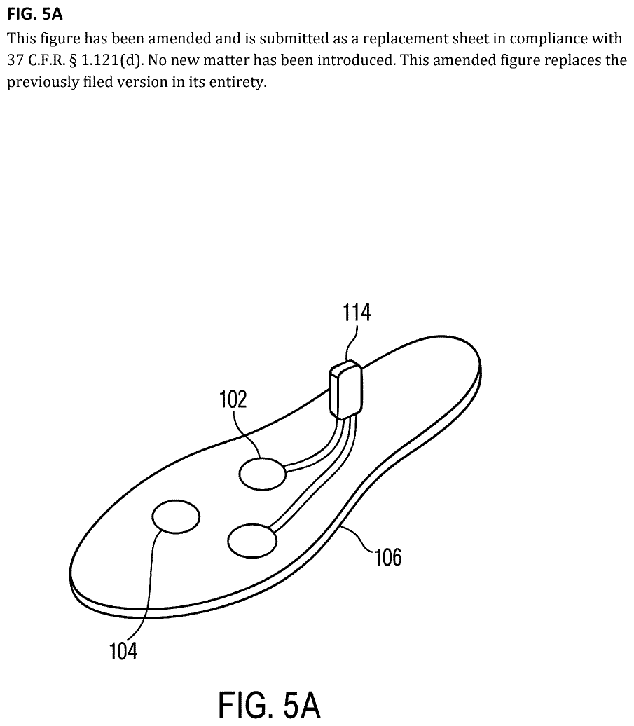

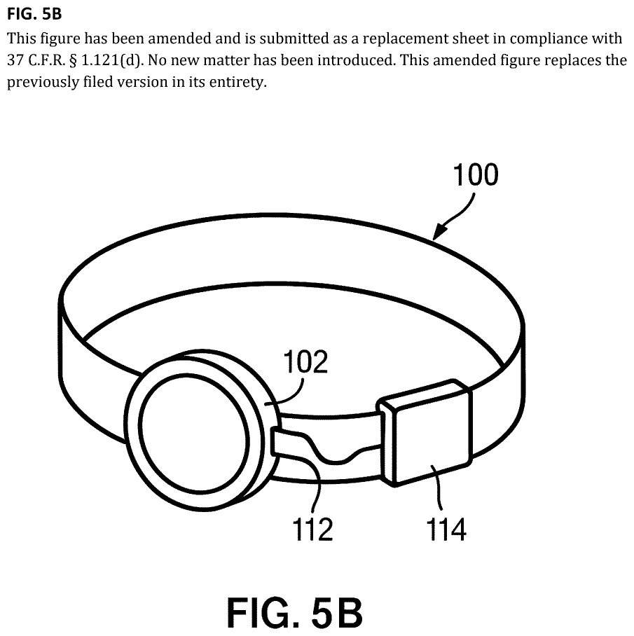

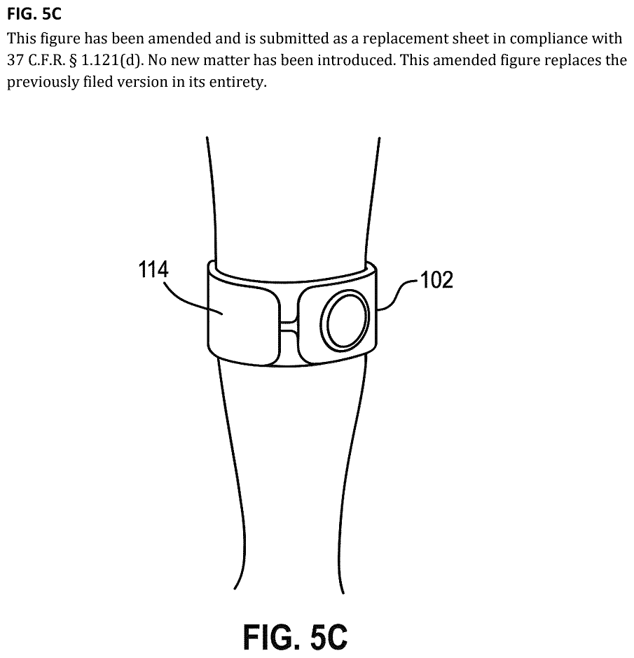

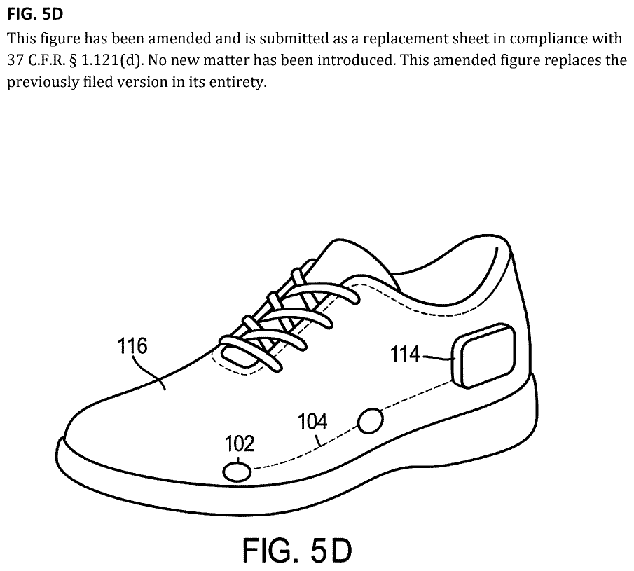

FIG. 5 illustrates at 500 the apparatus 100 embedded in different configurations, in accordance with an embodiment of the present disclosure. In various embodiments, the apparatus 100 may be used in a form of a wearable cuff, (for example, ankle cuffs), an adhesive patch, the insoles, waistbands, and in shoes. The apparatus 100 may be equipped with modular components such as a multiple set of sensors, actuators, and controllers. The different configurations may be designed to detect physiological or biomechanical parameters of the user in the different user modes of operation, and may be used to deliver targeted stimulation, for enhancing venous circulation in the limb of the user, based on user activity or environmental conditions, for example, in the zero-gravity environment and during no movement of the user.

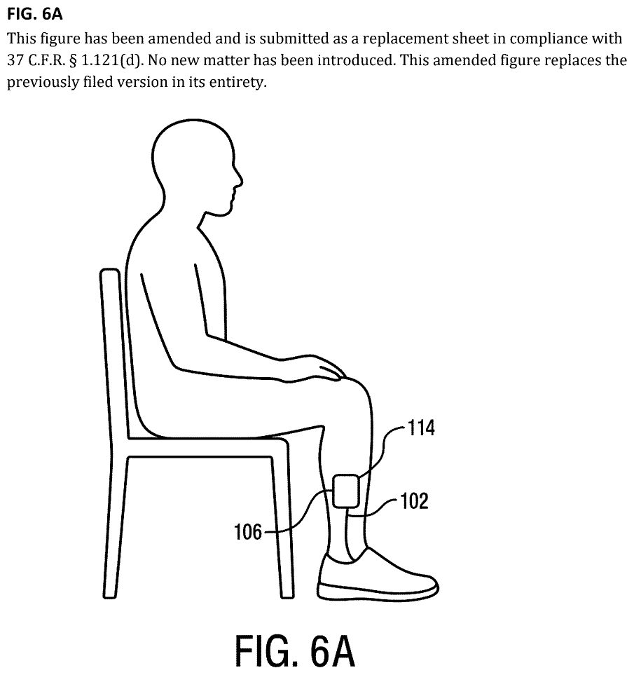

FIG. 6 illustrates, at 600, the apparatus 100 being used for enhancing the venous circulation in the limb of the user when the user is sitting, in accordance with an embodiment of the present disclosure. In an exemplary scenario and with respect to FIG. 6, the user may be seated on a chair. The user sitting on the chair may represent the passive user mode of operation. The passive user mode of operation may be related to, for example, prolonged sitting or being present in the zero-gravity environment. The user sitting on the chair may be wearing the footwear 300 and may have the apparatus 100 integrated within. The apparatus includes the first set of sensors 102, 104 and 106, the second set of sensors 108 and 110, the third set of sensors 112, and the haptic actuator 114 which may be used to promote the venous circulation in the limb of the user. A controller 602 may be mounted on the chair or specifically to a leg of the chair. The controller 602 may function as a processing source and may be connected wirelessly with each of the first set of sensors 102, 104 and 106, the second set of sensors 108 and 110, the third set of sensors 112, and the haptic actuator 114. In an exemplary embodiment, the haptic actuator 114 may be attached at the thigh or the calf of the user as a strap. The haptic actuator 114 may include multiple stimulation electrodes that may provide localized stimulation at the limb, to enhance muscle activity of the user resulting in improved venous circulation. With respect to the example, the chair may serve as a fixed reference frame when the user is present in a stationary seated position.

In continuation of the above-mentioned example, the apparatus 100 may be used as a removable insole and may be used for measuring the venous-pooling index. The venous-pooling index may be used for detection of the gait and for activating the haptic actuator 114. In addition, the apparatus 100 may be present as a band, which may be, for example, an adjustable elastic or a fabric band, that may be likely worn on a leg of the user (e.g., the thigh or the calf).

The controller 602 of the apparatus 100 may be a standalone controller or a wireless interface unit and may be responsible for processing sensor data, activating stimulation, and sending information to an external entity, for example, a doctor monitoring the user (e.g., the patient).

Thus, the apparatus 100 may be used to monitor physiological parameters (like the pressure or the movement) of the user and deliver haptic stimulation to enhance blood flow or muscle activity when the user is immobile or is in the active user mode of operation. This may help prevent conditions such as the venous pooling, the edema, or the DVT. The apparatus 100 may be particularly used in the zero-gravity environments which arise during space missions. Further, the apparatus 100 may be used in scenarios such as during clinical recovery, and the like.

FIG. 7 illustrates a method 700 for enhancing the venous circulation in the limb of the user, in accordance with an embodiment of the present disclosure. With respect to FIG. 7, at step 702, the first set of sensors are used to detect an amount of load exerted by the limb of the user. The first set of sensors are configured to be placed in contact with the limb of the user. At step 704, the second set of sensors are used for detecting a blood circulation level corresponding to the limb of the user. The second set of sensors are configured to be placed in contact with a portion of skin of the limb of the user to optically detect the blood circulation level. At step 706, the third set of sensors are used for detecting movement and orientation of the limb of the user. The third set of sensors are configured to be placed in contact with the limb of the user. At step 708, the haptic actuator is configured to be placed in contact with the limb of the user and is used to generate an external stimulation. Further, at step 710, the controller determines a venous-pooling index based on the amount of load that is exerted by the limb of the user, the blood circulation level corresponding to the limb of the user, and the movement and orientation of the limb of the user. The controller is configured to instruct the haptic actuator to generate the external stimulation for the limb when the venous-pooling index exceeds a pre-defined threshold, to enhance the venous circulation in the limb of the user.

FIG. 8A illustrates a block diagram 800 of a firmware architecture for a controller of the apparatus 200, including data acquisition, filtering, AI decision logic, and wireless communication layers, in accordance with an embodiment of the present disclosure.

With respect to FIG. 8A are shown four horizontal layers. At top is a data acquisition layer 808 which receives data from three sensor inputs, such as the pressure sensors (Load) 802, the optical sensors (PPG/NIRS) for Blood Circulation/Perfusion Index 804, the motion sensors 806 (IMU) for movement/orientation. Next is shown a filtering layer which contains sub-blocks such as Exponential Moving Average (EMA) and Kalman Filter. Next is the AI/ML decision layer 810 with sub-elements VPI/Stasis Score Calculation (VPI=w₁*(1-normalized_load)+w₂*(threshold-measured_blood_level/PI)+w₃*(immobility_duration/max_duration)), Machine Learning models for predictive stasis trends/personalization, and threshold check.

At bottom is a communication layer 812 with a Bluetooth Low Energy (BLE) wireless pairing/sync (for bilateral devices), Over The Air (OTA) firmware updates, and data transmission to mobile app/cloud (remote monitoring).

Solid arrows show downward data flow labeled real-time processing, and a dashed arrow shows optional cloud sync. An arrow from the AI/ML layer points to a haptic actuator (external stimulation).

FIG. 8B illustrates a block diagram 840 of a firmware architecture for a medical device aimed at improving venous circulation, in accordance with an embodiment of the present disclosure.

With respect to FIG. 8B the firmware architecture is represented to have four horizontal layers. A data acquisition layer 842 at top receives arrows from the pressure sensors (Load), the optical sensors (PPG/NIRS for blood circulation/perfusion index), and the motion sensors (IMU for movement/orientation)”. The filtering layer includes the EMA and the Kalman Filter. The AI/ML Decision layer 844 contains the VPI/stasis score calculation (VPI=w₁*(1−normalized_load)+w₂*(threshold measured_blood_level/PI)+w₃*(immobility_duration/max_duration)), the machine learning models for the predictive stasis trends/personalization, and the threshold check. The communication layer 846 at the bottom shows the BLE wireless pairing/sync (for the bilateral devices), the OTA firmware updates, and the data transmission to the mobile Application/the cloud (remote monitoring). The real-time processing arrow between the layers, the dashed arrow indicates the optional cloud synchronization. The output arrow indicates the haptic actuator (external stimulation).

FIG. 9 illustrates a flow chart 900 for calculating the venous-pooling index using a weighted algorithm and the machine learning model, in accordance with an embodiment of the present disclosure.

With respect to FIG. 9, at step 902, the sensor data from the pressure sensors (Load normalized 0-1), the optical sensors (PI=(AC/DC)×100%), the motion sensors (Immobility Duration) is received at an apply filtering (EMA/Kalman) block. At step 904, the weighted VPI is computed, while receiving data from the optical sensors. At step 906, the filtering is performed while receiving the personalized threshold data and the predicted stasis.

At step 908, the weighted VPI is computed as w₁*(1−normalized_load)+w₂*(threshold-PI)+w₃* (immobility_duration/max_duration) (weights: 0.4,0.4,0.2).

Further, at step 910, it is determined whether the ML model may be used for the trend prediction. In case, it is affirmative (yes), then a neural network on the historical data is applied to personalize the threshold/predict the stasis and in case of ‘No’ a bypass decision is taken. Both paths merge into a decision, at step 912 to determine whether VPI/Stasis Score>Threshold ?. The ‘Yes’ indicates Trigger Haptic Stimulation (1-5 Hz Vibration) and ‘No’ loops back to monitor sensors, at step 914. In addition, a dashed arrow loops from the monitor sensors back to start for continuous monitoring.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

1. An apparatus for enhancing venous circulation in a limb of a user, the apparatus
comprising:
a first set of sensors positioned under heel, arch, and forefoot regions of a foot, the first set of sensors configured to measure plantar loading associated with a plantar-venous-plexus foot-pump cycle;
a second set of sensors positioned beneath at least a first and second metatarsal head of the foot, the second set of sensors configured to optically detect perfusion through the plantar venous plexus and to compute a perfusion index (PI) derived from a photoplethysmographic (PPG) AC/DC signal;
a third set of sensors configured to detect movement, gait-phase transitions, and orientation of the limb;
a haptic actuator positioned beneath at least one metatarsal head and configured to deliver targeted mechanical stimulation to the plantar venous plexus; and
a controller in communication with the first, second, and third sensors and the haptic actuator, the controller configured to:
(1) determine a venous-pooling index using a weighted algorithm integrating plantar loading, perfusion index, and immobility duration;
(2) maintain, under zero-gravity conditions, a preload assembly configured to apply a contact force of 0.5-2 N to the first set of sensors to ensure continuous skin-sensor contact; and
(3) instruct the haptic actuator to deliver plantar-venous-plexus stimulation when the venous-pooling index exceeds a personalized threshold generated by at least one artificial-intelligence or machine-learning model.

2. The apparatus as claimed in claim 1, wherein the first set of sensors comprises one or more pressure sensors.

3. The apparatus as claimed in claim 2, wherein a preload assembly is configured to apply a contact force to the one or more pressure sensors which are placed in contact with the limb of the user, to capture pressure measurements corresponding to the limb in a zero-gravity condition.

4. The apparatus as claimed in claim 3, wherein under the zero-gravity condition, the contact force applied to the one or more pressure sensors ranges from 0.5 newtons to 2 newtons.

5. The apparatus as claimed in claim 3, wherein in a zero-gravity condition the preload assembly maintains 0.5-2 N contact force and the controller applies a machine-learning model trained on zero-gravity perfusion and motion data to personalize a stimulation threshold and pattern.

6. The apparatus as claimed in claim 1, wherein the controller is configured to operate in an active user mode, and a passive user mode associated with the user, wherein during the operation of active user mode, the controller is configured to augment voluntary movement of the user through the external stimulation generated by the haptic actuator, and
wherein during the operation of passive user mode, the controller is configured to activate the haptic actuator to generate the external stimulation when the user is immobile or when the user is present in the zero-gravity environment.

7. The apparatus as claimed in claim 1, wherein the second set of sensors comprises one or more optical sensors.

8. The apparatus as claimed in claim 1, wherein the second set of sensors is configured to compute a perfusion index (PI) as alternating current to direct current (AC/DC) of a photoplethysmogram (PPG) signal, and the PI is used to calculate the venous-pooling index.

9. The apparatus of claim 1, wherein the controller adjusts a stimulation frequency, amplitude, and duty cycle in real time as a function of the venous-pooling index and perfusion index, decreasing stimulation as perfusion improves, with ranges including 1-5 Hz, 0.1-2 g peak acceleration, and 10-60 s burst duration.

10. The apparatus as claimed in claim 1, wherein the third set of sensors comprises one or more motion sensors.

11. The apparatus as claimed in claim 1, wherein the limb is a lower limb of the user and corresponds to a foot of the user.

12. The apparatus as claimed in claim 11, wherein the haptic actuator is positioned under a first metatarsal head and a second metatarsal head of the foot of the user.

13. The apparatus as claimed in claim 1, wherein the apparatus is configured as any or a combination of a wearable cuff, a footwear, and a sock.

14. The apparatus as claimed in claim 1, wherein the controller is configured to determine the venous-pooling index using a weighted algorithm integrating the first, second, and third sensor data.

15. The apparatus as claimed in claim 1, wherein the controller incorporates at least one of an artificial intelligence (AI) or machine learning (ML) model to predict venous stasis trends and personalize the external stimulation.

16. The apparatus as claimed in claim 1, further comprising wireless communication capabilities, including Bluetooth Low Energy (BLE) for synchronization with a paired device on a limb to assess bilateral symmetry in the venous circulation.

17. The apparatus as claimed in claim 1, wherein the apparatus is configured in a modular form factor selected from a group consisting of an ankle cuff, an adhesive patch, a leg band, a calf strap, and a chair-mounted controller.

18. A method for enhancing venous circulation in a limb of a user, the method comprising:
detecting, by a first set of sensors, an amount of load exerted by the limb of the user, where the first set of sensors are configured to be placed in contact with the limb of the user;
detecting, by a second set of sensors, a blood circulation level corresponding to the limb of the user, where the second set of sensors are configured to be placed in contact with a portion of skin of the limb of the user to optically detect the blood circulation level;
detecting, by a third set of sensors, movement and orientation of the limb of the user, where the third set of sensors are configured to be placed in contact with the limb of the user, and generating, by a haptic actuator, an external stimulation, where the haptic actuator is configured to be placed in contact with the limb of the user; and
determining, by a controller, a venous-pooling index based on the amount of load that is exerted by the limb of the user, the blood circulation level corresponding to the limb of the user, and the movement and orientation of the limb of the user, and instructing, by the controller, the haptic actuator to generate the external stimulation for the limb when the venous-pooling index exceeds a pre-defined threshold, to enhance the venous circulation in the limb of the user.

19. The method as claimed in claim 18, wherein determining the venous-pooling index comprises applying a weighted algorithm to the detected data.

20. The method as claimed in claim 18, further comprising computing a stasis score as a weighted combination of ΔP (pressure trend), tinactive (inactivity duration), and (1−PI), and triggering haptic stimulation when the stasis score exceeds a predetermined level.
